(19) United States
(12) Patent Application Publication
(10) Pub. No.: US 2023/0241281 A1
(43) Pub. Date: Aug. 3, 2023

(54) PLASMA-BASED FLUID DISINFECTION DEVICE

(71) Applicants: Raj Kamal Singh, Columbia, PA (US); Hema Priyamvada Ravindran, Columbia, PA (US)

(72) Inventors: Raj Kamal Singh, Columbia, PA (US); Hema Priyamvada Ravindran, Columbia, PA (US)

(21) Appl. No.: 18/131,718

(22) Filed: Apr. 6, 2023

Related U.S. Application Data
(60) Provisional application No. 63/418,668, filed on Oct. 24, 2022.

(51) Int. Cl. A61L 9/22

(52) U.S. Cl. CPC A61L 9/22; A61L 2202/122; A61L 2202/11

(57) ABSTRACT

The disclosure provides a plasma-based cyclone fluid disinfection and filter device for the removal of particles and disinfection of fluid using a non-thermal plasma. The device comprises a housing configured to filter the fluid, comprising a side walls defining a primary cavity, and a non-thermal plasma reactor configured within the side walls to generate the non-thermal plasma within the side walls. Further, the device comprises an inlet opening within the side walls to enter the fluid within the side walls disinfect the inlet fluid within the side walls using the non-thermal plasma. Further, the device comprises a cylindrical inner tube connected with a fluid outlet, and a DBD plasma reactor defined within the walls of the cylindrical inner tube to further disinfect the fluid using the plasma, before exhausting it out within the environment. Further, the device comprises a detachable cassette and an ionizer.

### CROSS-REFERENCE TO RELATED APPLICATION

This application claims the benefit under 35 U.S.C. & 119(e) of a U.S. Provisional Application Ser. No. 63/418,668 filed on Oct. 24, 2022, which is hereby incorporated by reference in its entirety.

### TECHNICAL FIELD

The present invention relates to a non-thermal cyclone plasma disinfection device for a particle collection and disinfection of a fluid flow using a non-thermal plasma.

### BACKGROUND

The pandemic of covid-19 has given poor indoor air quality its first-ever significant moment in the spotlight. Earlier, there was no importance given to the measurement and enhancement of the air quality within a closed environment. Rampant disease transmission within the hospitals triggered by a contaminated environment heightened attention because airborne viruses spread much faster indoors than outdoors. However, the non-availability of any effective indoor air disinfecting technology has elevated the health risk and economic burden to patients, hospitals, and health insurance companies.

Conventionally, to purify the air, there exist some air purifiers. But such conventional air purifiers are only capable to remove the dust particles of micro-objects present within the air. While any microorganism such as a virus present within the air does not get trapped within the air purifier. Further, some conventional air purifiers use ultraviolet filters to destroy biological impurities or microorganisms. But there is a limitation with the ultraviolet filter such as a shorter residence time of the air within the filter. This shorter residence time prevents the complete elimination of the micro-organisms present within the air and some micro-organisms are still available within the filtered air.

Another effective technology that can be used to disinfect pathogens is a Non-thermal plasma (NTP). The NTP can disinfect the pathogens up to 7-log reduction within seconds to minutes of operations. Further, it can degrade gaseous pollutants such as volatile organic carbons (VOCs).

Air and fomite sterilization with NTP is rapid and can non-selectively destroy a wide variety of pathogens such as bacteria, fungi, spores, and viruses. Disinfection by NTP can be highly feasible as the reactive oxygen species (ROS) and the reactive nitrogen species (RNS) generated by the plasma can penetrate the cell surface of bacteria (cells and spore forms), fungi, and viruses and destroy the underlying bio-molecular components. Further, these ROS and RNS can degrade the VOCs to harmless gaseous molecules.

Although these benefits, the conventional plasma reactors are inefficient in pollutant removal at a realistic clean air delivering rate (CADR) because of their shorter residence time and exposure within the reactor.

Therefore, there exists a need for a cyclone plasma disinfection device that may enable the unfiltered disinfected air to travel in a cyclone motion and spend more time within the device. Further, there is a need for a cyclone plasma disinfection device that may completely eliminate the micro-organisms using the non-thermal plasma.

### SUMMARY AND OBJECTIVES

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The present disclosure discloses a non-thermal cyclone plasma disinfection device for a particle collection and disinfection of a fluid flow using a Non-thermal plasma.

An aspect of the present disclosure relates to a plasma-based fluid disinfection and filter device. The device, according to the present aspect, is provided with a cyclone separator housing configured to filter heavy particles out of the fluid and a non-thermal plasma reactor configured within the housing. The housing comprising side walls defining a primary cavity, a fluid inlet, and a fluid outlet. The housing is configured for a cyclone motion of the fluid within the side walls and the primary cavity of the housing. The non-thermal plasma reactor is configured within the side walls of the housing to disinfect the fluid using the plasma. The non-thermal plasma reactor is a dielectric-barrier discharge (DBD) plasma reactor or metal-to-metal corona discharge reactor.

The housing further comprises a top opening configured at a proximal end of the housing with a cover mounted on the top opening, and a bottom opening configured at a distal end of the housing. The side walls of the housing further comprises a primary layer and a coaxial secondary layer with a diameter lesser than the primary layer, creating a primary annulus guideway between the primary layer and the secondary layer of the side walls. The primary layer may selectively be made of a dielectric material, or a conductive metal and the secondary layer may respectively be made of the conductive metal or the dielectric material.

Further, the side walls may comprise a metal sleeve configured over an outer surface of the primary layer defining the DBD plasma reactor, if the primary layer is made of the dielectric material. Further, the side walls may comprise a metal sleeve configured over an inner surface of the secondary layer defining the DBD plasma reactor, if the secondary layer is made of the dielectric material. The layer of the side walls made of the conductive metal and the metal sleeve defines an electrode of the DBD plasma reactor.

Further, the electrodes may be connected with a high voltage power source to create the non-thermal plasma within the primary annulus guideway of the outer body.

Further, the device comprises a fluid inlet and a fluid outlet. The fluid inlet is configured near a proximal end of the housing and the fluid outlet configured within the cover mounted on the top opening of the housing. The fluid inlet opens within the primary annulus guideway of the side walls and the fluid outlet opens within the primary cavity within the housing.

The fluid inlet is configured to enter the fluid within the primary annulus guideway and travel spirally from the proximal end towards a distal end of the housing. The fluid travels in contact with the plasma present within the primary annulus guideway for disinfecting and filtering the fluid and creating an outer vortex of the fluid.

Further, the device comprises a detachable cassette mounted at the bottom opening of the housing. The detachable cassette comprises an upper layer made of a metal mesh, and a parallel lower layer made either of a metal sheet or the metal mesh. The detachable cassette is configured to couple with the high-voltage power source to create the plasma between the upper layer and the lower layer of the cassette to further disinfect the fluid. The detachable cassette is further configured as a collection unit for dust or particles separating from the fluid due to the cyclone motion.

The high voltage power source may be connected with the solid metal disk and the mesh plate of the detachable cassette as an electrodes to create a non-thermal plasma between the plates.

In another aspect, the disclosure relates to a plasma-based fluid disinfection and filter device that further comprises an elongated cylindrical inner tube coaxially mounted within the primary cavity and comprising a proximal end and a distal end, wherein the proximal end is connected with the fluid outlet, and the distal end opens within the primary cavity.

According to this aspect, the elongated cylindrical inner tube may further comprises a primary layer and a coaxial secondary layer with a diameter lesser than the primary layer, creating a secondary annulus guideway between the primary layer and the secondary layer of the cylindrical inner tube. Further, the primary layer of the cylindrical inner tube may be made of a dielectric material or a conductive metal and the secondary layer of the cylindrical inner tube may respectively be made of the conductive metal or the dielectric material.

Further, the cylindrical inner tube may comprise a metal sleeve configured over an outer surface of the primary layer defining a secondary DBD plasma reactor, if the primary layer is made of the dielectric material. Further, the cylindrical inner tube may comprise a metal sleeve configured over an inner surface of the secondary layer defining a secondary DBD plasma reactor, if the secondary layer is made of the dielectric material. The layer of the cylindrical inner tube made of the conductive metal and the metal sleeve defines an electrode of the secondary DBD plasma reactor.

Further, the electrodes may be connected with a high voltage AC or DC or pulsed DC power source to create the non-thermal plasma within the secondary annulus guideway of the cylindrical inner tube to further disinfect the fluid discharging out of the device.

Further, the conductive metal layers of the side walls and the cylindrical inner tube may be made either of a plain metal sheet, a corrugated metal sheet, a perforated metal sheet, a grated metal sheet, a wired mesh, a sheet made of a metal rod, or a spiked or nailed metal sheet.

Further, the device may comprise an ionizer configured within the fluid inlet to ionize the fluid entering within the device.

The object of the present disclosure is to provide a cyclone plasma-based fluid disinfection device that using the cyclone movement of the fluid, separates out the heavy particles such as dust present within the fluid. Further, the object is to increase the residence time of the fluid within the non-thermal plasma to completely disinfect the fluid before releasing again out in the environment.

### BRIEF DESCRIPTION OF DRAWINGS

The embodiments of the disclosure itself, as well as a preferred mode of use, further objectives, and advantages thereof, will best be understood by reference to the following detailed description of an illustrative embodiment when read in conjunction with the accompanying drawings. One or more embodiments are now described, by way of example only, with reference to the accompanying drawings in which:

FIG. 1A shows a cross-sectional view of a plasma-based cyclone fluid disinfection and filter device, according to first aspect of the present disclosure;

FIG. 1B shows a cross-sectional view of the plasma-based cyclone fluid disinfection and filter device, according to another embodiment of the first aspect of the present disclosure;

FIG. 2A shows a cross-sectional view of the plasma-based cyclone fluid disinfection and filter device, according to second aspect of the present disclosure;

FIG. 2B shows a cross-sectional view of the plasma-based cyclone fluid disinfection and filter device, according to another embodiment of the second aspect of the present disclosure;

FIG. 3A shows a cross-sectional view of the plasma-based cyclone fluid disinfection and filter device, according to an embodiment of the first aspect of the present disclosure;

FIG. 3B shows a cross-sectional view of the plasma-based cyclone fluid disinfection and filter device, according to another embodiment of the second aspect of the present disclosure;

FIG. 4 shows a cross-sectional view of the plasma-based cyclone fluid disinfection and filter device with an ionizer, according to an embodiment of the present disclosure;

FIG. 5 is a view showing simulation of the trajectory of the fluid within the device, according to an embodiment of the present disclosure.

### DETAILED DESCRIPTION

In the following description, numerous specific details are set forth in order to provide a thorough understanding of the embodiments of the present invention. It will be apparent to one skilled in the art that embodiments of the present invention may be practiced without some of these specific details.

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware and or by human operations.

If the specification states a component or feature “may”. “can”, “could”, or “might” be included or have a characteristic, that particular component or feature is not required to be included or have the characteristic.

As used in the description herein and throughout the claims that follow, the meaning of “a”. “an”, and “the” includes plural reference unless the context clearly dictates otherwise. Also, as used in the description herein, the meaning of “in” includes “on” unless the context clearly dictates otherwise.

Exemplary embodiments will now be described more fully hereinafter with reference to the accompanying drawings, in which exemplary embodiments are shown. This invention may, however, be embodied in many different forms and should not be construed as limited to the embodiments set forth herein. These embodiments are provided so that this invention will be thorough and complete and willfully convey the scope of the invention to that ordinary skill in the art. Moreover, all the statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

While embodiments of the present invention have been illustrated and described, it will be clear that the invention is not limited to these embodiments only. Numerous modifications, changes, variations, substitutions, and equivalents will be apparent to those skilled in the art, without departing from the spirit and scope of the invention, as described in the claim.

The disclosure discloses a plasma-based cyclone fluid disinfection and filter device provided to disinfect the inlet air or fluid using a non-thermal plasma as well as filter the inlet fluid using cyclone separation. The inlet fluid may be a polluted or infected air, gases, or a mixture of the same.

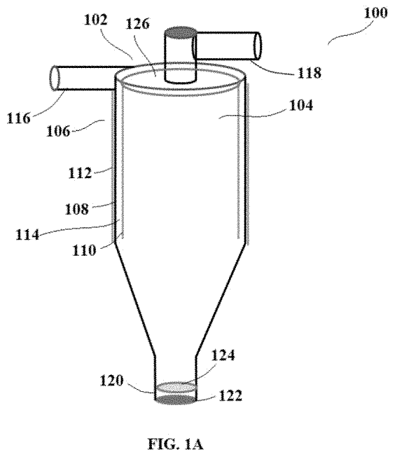

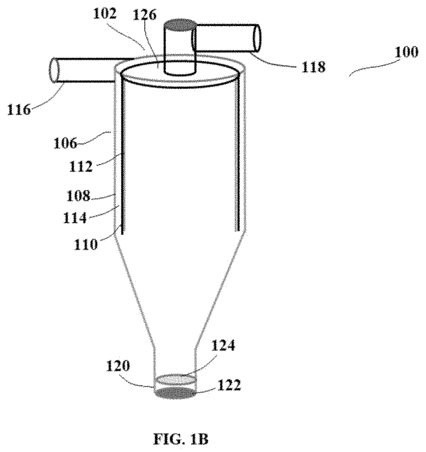

FIG. 1A shows a cross-sectional view of a plasma-based cyclone fluid disinfection and filter device 100, according to first aspect of the present disclosure.

Referring to FIG. 1, the plasma-based cyclone fluid disinfection and filter device 100, hereinafter may referred as a “disinfection device 100”, comprises a housing 102 having a side walls 106 defining a primary cavity 104, a fluid inlet 116, a fluid outlet 118, and a non-thermal plasma reactor defined within the side walls 106 of the housing 102. The housing 102 further comprises a top opening with a cover mounted at a proximal end of the housing 102 and a bottom opening configured at a distal end of the housing 102. Further, the disinfection device 100 comprises a detachable cassette 120 mounted on the bottom opening at a distal end of the housing 102.

Further, the side walls 106 of the housing 102 comprises plurality of layers including a primary layer 108, a coaxial secondary layer 110, and a primary annulus guideway 114 between the primary layer 108 and the secondary layer 110 that defines the dielectric-barrier discharge (DBD) plasma reactor within the side walls 106 of the housing 102. The coaxial secondary layer 110 comprises a diameter lesser than the diameter of the primary layer 108 that defines a primary annulus guideway 114 between the layers. Further, the side walls 106 comprises a metal sleeve 112 configured over the primary layer 108 defining a dielectric-barrier arrangement for a dielectric-barrier discharge (DBD) plasma reactor 106 using the primary layer 108, the secondary layer 110, and the metal sleeve 112.

According to an embodiment, the housing 102 may further comprises multiple sections including the elongated cylindrical upper section, a conical middle section, and a cylindrical bottom section. In another embodiment, the housing 102 may be made in a shape of an elongated cone.

Further, the primary layer 108 is made of a dielectric material and the secondary layer 110 is made of a conductive metal. For instance, the primary layer 108 may be made from any of a glass, quartz, ceramics, or polymers. The secondary layer 110 may be made from any of a copper, silver, aluminum, lead, platinum or alloy. Further, DBD plasma reactor defined within the side walls 106 comprises a metal sleeve 112 configured over the primary layer 108. The metal sleeve 112 may be a copper sleeve. The metal sleeve 112 and the secondary layer 110 of the side walls 106 are configured to behave as two electrodes of the plasma reactor 106, when connected with the high voltage power source (not shown). The electric discharge from the high voltage power source between the metal sleeve 112 and the secondary layer 110 working as two electrodes, separated by the dielectric insulating primary layer 108, creates a non-thermal plasma within the primary annulus guideway 114 between the primary layer 108 and the secondary layer 110. The high voltage power source may be any of a high voltage AC current source, a high voltage DC current source, or a high voltage pulsed DC current source.

Further, the fluid inlet 116 is configured within the side walls 106 near the proximal end of the housing 102. Further, the fluid inlet 116 is configured to open within the primary annulus guideway 114 of the side walls 106. The fluid inlet 116 is configured to inlet the contaminated or polluted fluid within the primary annulus guideway 114 to come in contact with the non-thermal plasma present within the primary annulus guideway 114.

Further, the housing 102 of the disinfection device 100 causes the inlet fluid to travel spirally within the primary annulus guideway 114. The inlet fluid enters within the primary annulus guideway 114 from the fluid inlet 116 and travels spirally from the proximal end towards a distal end of the housing 102. This spiral travel of the inlet fluid creates an outer vortex of the fluid within the primary annulus guideway 114. Further, this spiral travel increases a residence time of the inlet fluid in contact with the non-thermal plasma present within the primary annulus guideway 114. Thus, resulting in maximum disinfection of the inlet fluid using the non-thermal plasma.

Further, the detachable cassette disk 120 is mounted on a bottom opening at the distal end of the housing 102. The detachable cassette 120 is configured to seal the opening at the distal end. Further, the detachable cassette is configured as a collection unit to collect the particles separated from the fluid at a bottom of the housing 102. Further, the detachable cassette 120 is removable to clean the collected particles. Further, the closure of the bottom opening of the housing 102 directs the outer vortex of the fluid from the distal end towards the proximal end creating an axial inner vortex within the primary cavity 104 of the housing 102. The inner vortex is created at a center of the primary cavity 104. Further, the detachable cassette 102 is made of an upper layer 124 made of a metal mesh, and a parallel lower layer 122 made of the metal sheet. The lower layer 122 may also be made of a metal mesh.

In an embodiment, the lower layer 122 and the upper layer 124 may be coupled with the HV power source defining at least one layer as the high voltage electrode and another layer as the ground electrode. The high voltage discharge of a current between the electrodes creates a plasma within the detachable cassette 120, between the layers. The plasma created within the detachable cassette 120 may further disinfect the fluid being directed towards the proximal end from the distal end within the primary cavity.

Further, the disinfection device 100 comprises a covering plate 126 mounted at top opening at the proximal end of the housing 102. The covering plate 126 further comprises the fluid outlet 118 configured coaxially at a center of the covering plate 126. The fluid outlet 118 is configured to exhaust out the disinfected inner vortex of fluid from the device 100.

Further, the cyclone or spiral vortex of the inlet fluid within the side walls 106 causes the heavy particles present within the fluid such as dust particles, dirt, or any other heavy particles to separate out and collect at the bottom of the device 100. The inertia of the particles which is usually greater than the inertia of the inlet fluid causes the particles to separate out of the fluid.

In an embodiment, the plasma reactor may be a dielectric-barrier discharge (DBD), a pulsed/AC/DC corona discharge, or a micro-discharge plasma reactor. Further, the electrodes of the reactor may be charged from 1 kV to several kV. For instance, the electrode of the reactor may be charges from 1 kV to the 50 kV.

FIG. 1B shows a cross-sectional view of the plasma-based cyclone fluid disinfection and filter device 100, according to another embodiment of the first aspect of the present disclosure.

According to this embodiment, the disinfection device 100 comprises a housing 102 comprising a DBD plasma defined within a side walls 106 of the housing 102 using plurality of layers. The disinfection device 100 comprises a primary layer 108, a coaxial secondary layer 110 having a lesser diameter than the primary layer 108, and a metal sleeve configured on inner surface of the secondary layer.

Further, according to this embodiment, the primary layer 108 may be made of any conductive metal including, but not limited to, the copper, Aluminum, Silver, lead, or Titanium. Further, the secondary layer may be made of a dielectric material including, but not limited to, a glass, quartz, ceramics, or polymers. Further, the metal sleeve 112 is mounted on the inner surface of the secondary dielectric layer 112 to define a dielectric barrier configuration of the DBD plasma reactor 106 between the primary layer 108 and the metal sleeve 112. Further, the primary conductive layer 108 and the metal sleeve layer 112 when connected with the high voltage power source, create a non-thermal plasma within the primary annulus guideway 114 between the primary conductive layer 108 and the secondary dielectric layer 110.

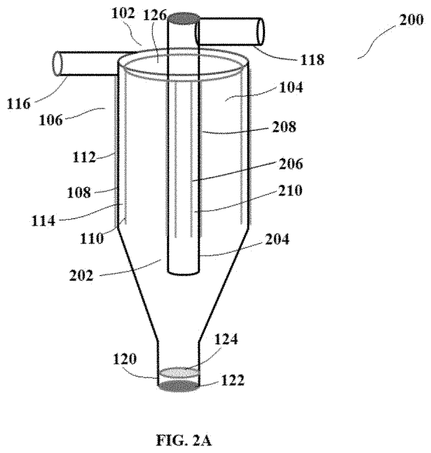

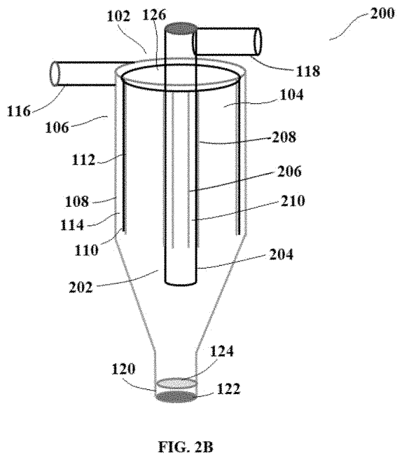

FIG. 2A shows a cross-sectional view 200 of the plasma-based cyclone fluid disinfection and filter device 100, according to second aspect of the present disclosure. FIG. 2B shows a cross-sectional view of the plasma-based cyclone fluid disinfection and filter device 100, according to another embodiment of the second aspect of the present disclosure.

Referring to FIG. 2A, according to the second aspect, the disinfection device 100 may comprise all the components similar to the embodiment of the FIG. 1A. Additionally, the disinfection device 100 may comprise an elongated cylindrical inner tube 202 coupled with the fluid outlet 118 and mounted within the primary cavity 104. The cylindrical inner tube 202 comprises a proximal end and a distal end. The proximal end is connected with the fluid outlet 118, whereas the distal end is open within the primary cavity. Further, the cylindrical inner tube 202 is mounted on an axial center of the primary cavity aligning with an axial inner vortex to enable the fluid to enter within the cylindrical inner tube 202 and exhaust out through the fluid outlet 118.

The cylindrical inner tube 202 further comprises a primary layer 204, and a coaxial secondary layer 206 inside the primary layer 204. The secondary layer 206 comprises a diameter lesser than the diameter of the primary layer 204 defining a second annulus guideway 210 between the primary layer 204 and the secondary layer 206 of the cylindrical inner tube 202.

Further, the primary layer 204 of the cylindrical inner tube 202 may be made of a dielectric material. Further, the secondary layer 206 may be made of a conductive metal such as copper. The cylindrical inner tube 202 further comprises a metal sleeve 208 configured over the dielectric primary layer 204 to create a dielectric barrier arrangement for the plasma reactor. Further, the device 100 may comprises a high voltage power source applied to the metal secondary layer 206 and the metal sleeve 210 as electrodes to define a third DBD plasma reactor and generate a non-thermal plasma within the secondary annulus guideway 210.

The third DBD plasma reactor defined within the cylindrical inner tube 202 is configured to further disinfect the inner vortex of fluid using the cold plasma before exhausting out into the environment.

Therefore, the device 100 comprises multiple plasma reactors to disinfect the inlet air multiple times in order to completely remove the microorganisms, pathogens, bacteria, etc. from the infected inlet air.

According to one embodiment of the second aspect, the housing 102 may comprise a plurality of layers defining the DBD plasma reactor within the side walls 106. The plurality of layers includes a primary layer 108 made of a dielectric material, a coaxial secondary layer 110 inside the primary layer 108 made of a conductive metal, and a metal sleeve 112 configured over the primary layer 108 as shown in the FIG. 2A.

According to another embodiment of the second aspect, the outer body 102 may comprise a primary layer 108 made of a conductive material such as metal, a coaxial secondary layer 110 made of the dielectric material, and the metal sleeve configured over an inner surface of the secondary layer 110 to define the DBD plasma reactor, as shown in the FIG. 2B.

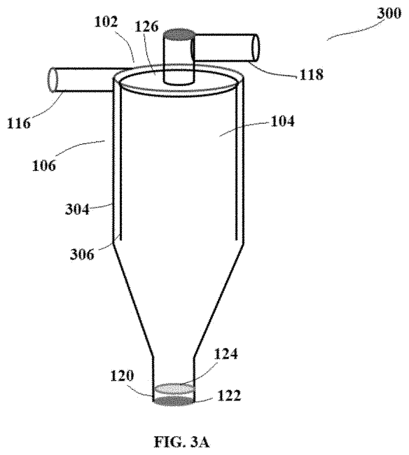

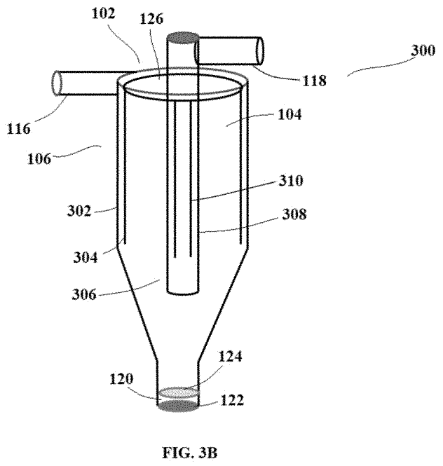

FIG. 3A shows a cross-sectional view 300 of the plasma-based cyclone fluid disinfection and filter device 100, according to an embodiment of the first aspect of the present disclosure. FIG. 3B shows a cross-sectional view 300 of the plasma-based cyclone fluid disinfection and filter device 100, according to another embodiment of the second aspect of the present disclosure;

Referring to FIG. 3A, the disinfection device 100 may comprise a metal-to-metal corona discharge plasma reactor defined within a side walls 106 of the housing 102. The side walls 106 may comprise a primary layer 302 and a coaxial secondary layer 304 made of a conductive metal. Further, the walls 106 may comprise an annulus guideway between the primary layer 302 and the secondary layer 304 defining the metal-to-metal discharge plasma reactor within the side walls 106. Further, the primary layer 302 and the secondary layer 304 may be connected as electrodes with the HV power source to generate a non-thermal plasma within the annulus guideway.

The disinfection device 100 may further comprise an elongated cylindrical inner tube 306 connected with the fluid outlet 18 and configured within the primary cavity 104, wherein the cylindrical inner tube 306 further comprises a primary layer 308 and a secondary layer 310 made of a conductive metal defining the metal-to-metal discharge plasma reactor within the cylindrical inner tube 306, as shown in the FIG. 3B.

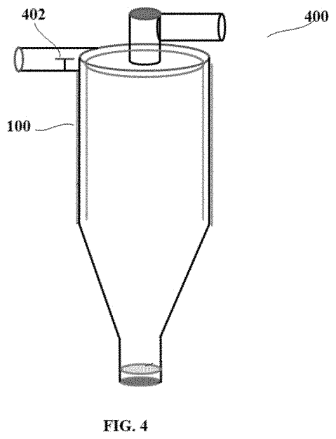

FIG. 4 shows a cross-sectional view 300 of the plasma-based cyclone fluid disinfection and filter device 100 with an ionizer 302, according to an embodiment of the present disclosure.

Referring to the FIG. 4, the disinfection device 100 further comprises an ionizer 402 configured within the inlet 116 of the device 100. The ionizer 402 is configured to ionize the tiny particles present within the inlet fluid to clump together and land on an internal surface of the housing 102. The ionizer 402 may be configured either horizontally, vertically, or laterally to a direction of inlet fluid flow, within the inlet 106. Further, the ionizer 402 may be configured anywhere within the housing 102 of the device 100.

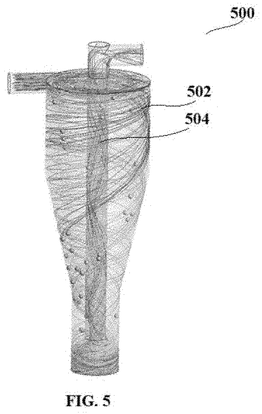

FIG. 5 is a view 500 showing simulation of the trajectory of the fluid within the device 100, according to an embodiment of the present disclosure.

Referring to FIG. 5, the inlet fluid entering from the inlet 116 moves into the guideway or primary annulus guideway in a cyclonic movement increasing the residence time of the inlet fluid inside the plasma filled guideway 114. Further, the cyclone movement of the inlet fluid from the proximal end towards the distal end of the device 100 creates an outer fluid vortex 502 within the annulus guideway 114. Further, the movement of the fluid from the distal end towards to proximal end creates an axial inner fluid vortex 504 within the primary cavity 104 of the device 100.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

Similarly, structures and functionality presented as a single component may be implemented as separate components. These and other variations, modifications, additions, and improvements fall within the scope of the subject matter herein. In addition, the term “each” used in the specification does not imply that every or all element in a group need to fit the description associated with the term “each”. For example, “each member is associated with element A” does not imply that all members are associated with element A. Instead, the term “each” only implies that a member (of some of the members), in a singular form, is associated with an element A.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that are issued on an application based hereon. Accordingly, the disclosure of the embodiments is intended to be illustrative, but not limiting, of the scope of the patent rights.

1. A plasma-based cyclone fluid disinfection and filter device, comprising:
a housing having side walls defining a primary cavity, a fluid inlet, and a fluid outlet, wherein the housing is configured for a cyclone motion of the fluid within the side walls and the primary cavity of the housing; and
a non-thermal plasma reactor configured within the side walls to disinfect the fluid using the plasma.

2. The device of claim 1, wherein the housing further comprises
a top opening configured at a proximal end of the housing with a cover mounted on the top opening; and
a bottom opening configured at a distal end of the housing.

3. The device of claim 1, wherein the side walls of the housing further comprise a primary layer and a coaxial secondary layer with a diameter lesser than the primary layer, defining a primary annulus guideway between the primary layer and the secondary layer of the side walls.

4. The device of claim 3, wherein the primary layer may selectively be made of a dielectric material or a conductive metal and the secondary layer may respectively be made of the conductive metal or the dielectric material.

5. The device of claim 4, wherein the side walls of the housing may further comprises
a metal sleeve configured over an outer surface of the primary layer defining a DBD plasma reactor, if the primary layer is made of the dielectric material; or
a metal sleeve configured over an inner surface of the secondary layer defining the DBD plasma reactor, if the secondary layer is made of the dielectric material.

6. The device of claim 5, wherein a layer of the side walls made of the conductive metal and the metal sleeve defines electrodes of the DBD plasma reactor.

7. The device of claim 6, wherein the electrodes are connected with a high voltage power source to create the non-thermal plasma within the primary annulus guideway of the side walls.

8. The device of claim 7, wherein
the fluid inlet is configured within the side walls near a proximal end of the housing; and
the fluid outlet is configured within the cover mounted on the top opening of the housing,
wherein the fluid inlet opens within the primary annulus guideway of the side walls and the fluid outlet opens within the primary cavity of the housing.

9. The device of claim 8, wherein the fluid inlet is configured to enter the fluid within the primary annulus guideway and travel in cyclone motion from the proximal end towards a distal end of the housing in contact with the plasma present within the primary annulus guideway.

10. The device of claim 9, further comprises a detachable cassette mounted at the bottom opening of the housing, wherein the detachable cassette comprises
an upper layer made of a metal mesh; and
a parallel lower layer made either of a metal sheet or the metal mesh.

11. The device of claim 10, wherein the detachable cassette is configured to couple with the high voltage power source to create the plasma between the upper layer and the lower layer to further disinfect the fluid.

12. The device of claim 11, wherein the detachable cassette is further configured as a collection unit for dust or particles separating from the fluid due to the cyclone motion.

13. The device of claim 12, further comprises
an elongated cylindrical inner tube coaxially mounted within the primary cavity and comprising a proximal end and a distal end, wherein
the proximal end is connected with the fluid outlet, and
the distal end opens within the primary cavity.

14. The device of claim 13, wherein the elongated cylindrical inner tube may further comprise a primary layer and a coaxial secondary layer with a diameter lesser than the primary layer, defining a secondary annulus guideway between the primary layer and the secondary layer.

15. The device of claim 14, wherein the primary layer of the cylindrical inner tube may be made of a dielectric material or a conductive metal and the secondary layer of the cylindrical inner tube may respectively be made of the conductive metal or the dielectric material.

16. The device of claim 15, wherein
the cylindrical inner tube may further comprises a metal sleeve configured over an outer surface of the primary layer defining a secondary DBD plasma reactor, if the primary layer is made of the dielectric material; or
the cylindrical inner tube may further comprises a metal sleeve configured over an inner surface of the secondary layer defining a secondary DBD plasma reactor, if the secondary layer is made of the dielectric material.

17. The device of claim 16, wherein
a layer of the cylindrical inner tube made of the conductive metal and the metal sleeve defines an electrode of the secondary DBD plasma reactor; and
the electrodes may be connected with a high voltage power source to create the non-thermal plasma within the secondary annulus guideway of the cylindrical inner tube to further disinfect the fluid.

18. The device of claim 17, wherein the electrodes may be charged between 1 kV to 50 kV.

19. The device of claim 18, wherein the conductive metal layers of the side walls and the cylindrical inner tube may be made either of a plain metal sheet, a corrugated metal sheet, a perforated metal sheet, a grated metal sheet, a wired mesh, a sheet made of a metal rod, or a spiked or nailed metal sheet.

20. The device of claim 1, further comprises an ionizer configured within the fluid inlet to ionize the particles in the fluid entering within the device.
